(19) United States
(12) Patent Application Publication
(10) Pub. No.: US 2014/0276284 A1
(43) Pub. Date: Sep. 18, 2014

(54) Tube Set with Anti Occlude Strip for Air Bladder Medical Devices

(71) Applicant: Compression Therapy Concepts, Inc., Eatontown, NJ (US)

(72) Inventors: Orlando Mansur, JR., Eatontown, NJ (US); Leonard Nass, Eatontown, NJ (US)

(21) Appl. No.: 14/216,616

(22) Filed: Mar. 17, 2014

Related U.S. Application Data
(60) Provisional application No. 61/786,413, filed on Mar. 15, 2013.

(51) Int. Cl. A61H 9/00; A61H 1/00; A61M 25/00

(52) U.S. Cl. CPC A61H 9/0078; A61M 25/0043; A61H 1/008
USPC 601/151; 604/523; 604/500

(57) ABSTRACT

An insertable anti-occlude strip includes a strip made of a material that is flexible, sturdy, durable and compression resistant. The strip has a cross-sectional shape such that when the strip is inserted into a hollow, flexible tube, there are open longitudinal apertures formed between the strip and the inner wall of the tube, allowing bi-directional flow of air or fluids through the tube. The insertable and tubal anti-occlude strip, when inserted into a hollow and flexible tube, provides resistance to occlusion of the tube lumen when the tube is under compressive or shear forces, which would otherwise significantly reduce or halt flow of air or fluids through the tube.

### RELATED APPLICATION

This application claims the benefit of priority to U.S. Provisional Application No. 61/786,413, filed on Mar. 15, 2013, entitled “Tube Set with Anti Occlude Strip For Air Bladder Medical Devices”, and currently co-pending.

### FIELD OF THE INVENTION

The present invention relates generally to medical and therapy devices. The present invention is more particularly useful as an insertable device for use in the prevention of the occlusion of medical and therapy device tubes. The present invention is particularly useful to prevent the occlusion of flexible medical and therapy device tubes during periods when a patient or attendant is unable to monitor proper function of the medical device.

### BACKGROUND OF THE INVENTION

Within the field of medicine a wide variety of devices are utilized to perform many different medically related tasks. Many of these tasks involve delivery of or removal of a fluid (i.e. air, blood, urine, saline) to or from a patient. Some examples are urine collection, blood withdrawal and intravenous rehydration with saline solution. The medical devices used in these situations typically require a direct connection from the device to the patient in the form of a tube. Depending on their intended use, the tube designs can vary widely in characteristics such as size (length, inner and outer diameter), hardness, flexibility, compressibility, durability, and biocompatibility. These characteristics are dictated by the choice of material from which the tube is formed, with polyvinylchloride (PVC), polyurethane (PU), silicone and latex rubber being quite commonplace.

One of the most critical concerns whenever a tube is used in a medical device is that its lumen remains patent. If the lumen decreases or collapses, the transmission of the fluid slows or ceases, presenting in many circumstances a medical emergency or at least a situation of medical concern. Causes of decreased lumen patency are numerous such as accumulation of mineral salts at the entrance to a urinary catheter or platelet aggregation at the exit of an intravenous catheter. Other causes are occlusion due to physical folding of or compression of the tube.

In a majority of medical device applications, a key characteristic of the tube is flexibility. It needs to be moveable and maneuverable to facilitate connection from the device to the patient, as device, patient location and position can vary widely. However, by increasing tubal flexibility, usually by adding plasticizers or other known additives, there is a softening effect in the composite material, which also increases compressibility, and thereby increases the occlusion potential of the tubal lumen. One way this occluding can happen is if the patient accidently compresses the device tube by lying on, sitting on or rolling over on it, or by bending a segment of the flexible tubing too far, thus pinching the lumen closed. Manufacturing a thicker tube wall can help remedy these situations, but brings added cost and decreased tubal flexibility. Additionally, smaller diameter tubes still remain flexible and tend to have fewer problems associated with bending and compression of the lumen, but are not suited for all situations, especially those situations requiring the transfer of lower density fluids at higher flow rates, as in the case of air transmission.

Intermittent pneumatic compression therapy (“IPC”) as a preventive treatment for deep vein thrombosis (“DVT”) incorporates the use of flexible tubes transferring air from an air pump to inflate and deflate airtight garments wrapped around a patient's limb. The successive inflations and deflations simulate the series of compressions applied to the limb veins during normal muscle contractions, and thereby limit any blood stasis that could lead to the formation of clots (“thrombi”). IPC can be of benefit to patients deemed to be at risk of deep vein thrombosis during extended periods of inactivity, and is an accepted treatment method for preventing blood clots or complications of venous stasis in persons after physical trauma, orthopedic surgery, neurosurgery, or in disabled persons who are unable to walk or mobilize effectively. This technique is also used to stop blood clots from developing during surgeries that will last for an extended period of time.

Complications from use of the IPC device can arise particularly if the airtight garments around the patient's limb do not deflate, leaving a prolonged state of increased pressure on the limb. This tourniquet-like effect can impede normal blood flow thus creating other problems such as swelling and improper tissue oxygenation toward the end of the limb. This improper deflation of the IPC garment can occur if the tube from the air pump to the inflatable garment is occluded, such as can happen if a patient accidentally lies or sits on the tube or the tube gets inadvertently compressed or bent as can happen when the patient is sleeping, for example. Some tubes in the industry are manufactured with a denser, less compressible material, usually metal, coiled within or lined the inside of their walls to prevent collapse or occlusion of the tubal lumen. These tubes can be effective in restricting occlusion; however, they are significantly more complex in design and require more costly manufacturing and production processes.

In light of the above, it would be advantageous to provide an anti-occlude strip that helps the tubes used in medical and therapy devices maintain its patency. It would be further advantageous to provide a tubal anti-occlude strip that can be easily customized for use in tubes of varied lengths and sizes, and that can be easily inserted into tubes of the types and grades already commonly used in the medical and therapy industries. It would be further advantageous to provide an anti-occlude strip that is easy to use and customize, relatively easy to manufacture, and comparatively cost efficient, for medical and therapy device tubes.

### SUMMARY OF THE INVENTION

The insertable, tubal anti-occlude strip (hereafter referred to as “anti-occlude strip”) of the present invention includes a strip made of a material that is flexible, sturdy, durable and compression resistant. The strip has a cross-sectional area with a shape that will allow fluids to continue to flow through a hollow tube when the strip has been inserted into the lumen of the tube. For instance, anti-occlude strip of the present invention may be formed with a major dimension slightly less than the interior diameter of the lumen of the hollow tube, and a minor dimension that creates longitudinal apertures that allow fluids to pass through the hollow tube. These apertures are created between the strip and the inner wall of the tube or within the strip itself to allow bidirectional fluid flow. Additionally, the anti-occlude strip preserves patency of the tube under external compressive or shear forces, or bending or kinking, which would otherwise occlude the tube if the anti-occlude strip of the present invention were not present.

In use, the anti-occlude strip of the present invention is sized to a length determined by the application, and inserted lengthwise into the lumen of a fluid-carrying, medical or therapy device tube. The tube containing the anti-occlude strip of the present invention is then connected to the medical or therapy device for standard device operation.

One application of the anti-occlude strip of the present invention is in an intermittent pneumatic compression (“IPC”) therapy device used for the prevention of deep vein thrombosis (“DVT”). An IPC device consists of an air pump, a compression garment that wraps around a patient's limb, and one or more medical-grade air tubes that connect the pump to the garment. The pump forces air through the air tube to inflate and deflate the compression garment thereby assisting in moving blood through the limb to prevent stasis and risk of DVT. The air tube connects to the pump and garment via connectors standard in the industry.

In use, a connector on one end of the air tube is removed. The anti-occlude strip of the present invention is sized lengthwise similar to the length of the IPC device air tube, and inserted the entire length of the tube. The connector is replaced on the air tube, and connected to the IPC device. Operation of the IPC device with its timed inflations and deflations begins, when the air tube, connecting the air pump and the compressive garment on the patient's limb are ready to be used for medical therapy purposes.

The length, the cross-sectional diameter and shape, and the material of the anti-occlude strip of the present invention are determined by the medical or therapy device tube characteristics as well as those of a particular application.

### BRIEF DESCRIPTION OF THE DRAWINGS

The nature, objects, and advantages of the present invention will become more apparent to those skilled in the art after considering the following detailed description in connection with the accompanying drawings, in which like reference numerals designate like parts throughout, and wherein:

FIG. 1 is a diagrammatic view of a preferred application for the anti-occlude strip of the present invention showing an IPC therapy device for the prevention of DVT with an air pump connected via a flexible, hollow air tube to a compressive garment on a patient;

FIG. 2 is a diagrammatic view of the IPC therapy device air pump connected to the flexible, hollow air tube with connectors, containing the anti-occlude strip of the present invention within the tube;

FIG. 3 is a cross-sectional view of the air tube of the IPC therapy device showing the anti-occlude strip of the present invention within the lumen of the tube as taken along line 3-3 of FIG. 2;

FIG. 4A is a diagrammatic view of the flexible air tube of the IPC therapy device in a bent configuration without the anti-occlude strip of the present invention inside the flexible air tube lumen;

FIG. 4B is a diagrammatic view of the flexible air tube of the IPC therapy device in a bent configuration equipped with the anti-occlude strip of the present invention inside the flexible air tube lumen;

FIG. 5A is a cross-sectional view of the flexible air tube of the IPC therapy device in a bent configuration without the anti-occlude strip of the present invention inside the flexible air tube lumen, as taken along line 5A-5A of FIG. 4A;

FIG. 5B is a cross-sectional view of the flexible air tube of the IPC therapy device in a bent configuration with the anti-occlude strip of the present invention inside the flexible air tube lumen, as taken along line 5B-5B of FIG. 4B;

FIG. 6 is a cross-sectional view of an alternative embodiment of the anti-occlude strip of the present invention formed with an anti-occlude strip in the shape of a “Y,” inserted within an air/fluid-carrying medial therapy tube having an outer wall, an inner wall, and a lumen;

FIG. 7 is a cross-sectional view of an alliterative embodiment of the anti-occlude strip of the present invention formed with an anti-occlude strip having a triangular shape, inserted within the lumen of a medical therapy tube having an outer wall and an inner wall;

FIG. 8 is a cross-sectional view of an alternative embodiment of the anti-occlude strip of the present invention in a rhomboidal or diamond-shape, located within the lumen of a medical therapy tube having an outer wall and an inner wall;

FIG. 9 is a cross-sectional view of an alternative embodiment of the anti-occlude strip of the present invention in a five-pointed, star-shape, located inside a lumen of a medical therapy tube having an outer wall and an inner wall; and

FIG. 10 is a diagrammatic view of an alternative embodiment of the anti-occlude strip of the present invention, when it is applied on a patient's hand, to connect the intravenous catheter which is inserted into a vein in the back of the patient's hand and a medical device.

### DETAILED DESCRIPTION OF A PREFERRED EMBODIMENT

Referring to FIG. 1, a view of a preferred application for the anti-occlude strip of the present invention is shown. Specifically, an IPC therapy device for the prevention of DVT consisting of an air pump 108 connected via a flexible, hollow air supply tube 110 to a compressive garment 106 positioned on the lower leg 104 of patient 102 is shown. The IPC therapy adopts a device configuration known in the industry. Pump 108 inflates and deflates compressive garment 106 by supplying pressurized air through flexible air supply tube 110 in directions 112 and 114, respectively. The anti-occlude strip 116 of the present invention is positioned within the hollow lumen of flexible air supply tube 110, and is shown in detail in FIG. 2 and FIG. 3. Flexible air supply tube 110 is made of common medical-grade tubing known in the industry, and is relatively transparent.

FIG. 2 is an enlarged view of the air pump 108 and flexible air supply tube 110 of the IPC therapy device shown in FIG. 1. Air supply tube 110 is equipped with quick-disconnect connectors 118 known in the industry to facilitate the changing of multiple devices, such as the compressive garment 106 (shown in FIG. 1), and air pump 108. The anti-occlude strip 116 of the present invention is sized to a length approximately that of the flexible air supply tube 110, and inserted into the lumen of air supply tube 110 by removing one of the quick-disconnect connectors 118 and inserting it through the opened end. The connector 118 is then replaced into air supply tube 110, and reconnected to compressive garment 106. Bi-directional air flow between pump 108 and compressive garment 106 occurs through apertures formed between anti-occlude strip 116 and the inner wall of air tube 110. Details of the apertures are shown in the FIG. 3 cross-sectional view.

Flexible air supply tube 110 having a non-descript length is shown. It is to be appreciated that the length of the air supply tube 110, and thereby the length of anti-occlude strip 116 of the present invention, may vary depending on the particular field of use, and the setting. For instance, in a hospital surgery setting, it may be difficult to position an air source immediately adjacent to the patient, and an extended air supply tube 110 is required.

The anti-occlude strip 116 of the present invention must be flexible, sturdy, durable and compression resistant, and must be relatively easy to cut, so its length can be customized for specific applications without difficulty. A material that maintains good tensile and compressive strength characteristics would be optimal. Some general examples of elastic polymers for use in the anti-occlude strip 116 of the present invention are vulcanized natural rubber, poly-isoprene (synthetic), and styrene-butadiene rubber. In a preferred embodiment, the anti-occlude strip of the present invention is manufactured using extrusion processes for cost minimization. It is to be appreciated that to those skilled in the art there may be materials and manufacturing processes known as well as shapes of anti-occlude strip 116, which might be most advantageous for a given patient application.

Referring now to FIG. 3, a cross-sectional view of the anti-occlude strip 116 of the present invention positioned within air supply tube 110 as taken along line 3-3 of FIG. 2 is shown. The industry standard medical-grade tubing 110 has a solid, circular cross-sectional area 123, an outer wall 122 and an inner wall 120 creating a hollow lumen 127 in which the anti-occlude strip 116 of the present invention is centrally positioned. Apertures 124A, 124B, 124C and 124D are hollow and patent, and formed between the anti-occlude strip 116 of the present invention and the inner wall 120 of the air supply tube 110. Anti-occlude strip 116 has a solid cross-sectional area 126, and an overall width less than an inner diameter 128 of the air supply tube 110 to provide for easy insertion of the anti-occlude strip 116 of the present invention into the air supply tube 110.

In a preferred embodiment, the shape of the cross-sectional area 126 of the anti-occlude strip 116 is cross-shaped. The cross shape provides sufficient resistance to compression and over-bending of the air supply tube 110, from any direction, to maintain patency, and provides sufficient airflow 112 and 114 through longitudinal apertures 124A, 124B, 124C and 124D to inflate and deflate, respectively, compressive garment 106 (shown in FIG. 1).

FIGS. 4A and 4B show air supply tube 110 in a severely bent configuration without and with the anti-occlude strip 116 of the present invention inserted into the air supply tube, respectively. It is to be appreciated that such bending of medical and therapy device tubes can readily occur in regular use applications due to the flexibility in the tube coupled with the medical attendant's misplacement or inadvertent patient's movement.

First, referring to FIG. 4A, air supply tube 110 is in a bent configuration creating a fold 130 in the air supply tube 110. Air supply tube 110 is shown with outer wall 122, inner wall 120 (represented by dashed lines), and a tubal lumen 132, which will be detailed in a cross-sectional view in FIG. 5A. The bending of air supply tube 110 creates a compression of the outer 122 and inner 120 tube walls, and thereby a significant narrowing of the tubal lumen 132 at the location of the fold 130. As bending of the air supply tube 110 increases from a normal, unstressed, straight configuration, progressive narrowing of the lumen 132 reduces, and at some point restricts, bi-directional air flow 112 and 114 through air supply tube 110. This can lead to a situation of medical concern if the compressive garment 106 remains pressurized around the patient's limb 104 (shown in FIG. 1) and is unable to deflate, thus restricting proper blood flow through the limb. It can be appreciated that in cases where tube 110 is bent significantly, the opening 134 in tube 110 can be completely closed, and the air passage through lumen which may cause catastrophic medical emergencies and patient harm.

Now referring to FIG. 4B, the anti-occlude strip 116 of the present invention inserted inside the inner wall 120 (represented by dashed lines) of air tube 110, is shown. In FIG. 4B, the anti-occlude strip 116 of the present invention and air supply tube 110, having outer wall 120 and inner wall 122, are in a bent configuration with a fold 130. Structural support from the anti-occlude strip 116 restricts compression of the air supply tube 110 at fold 130, preserving patency of the air supply tube 110, and allowing bi-directional airflow 112 and 114 to continue uninterrupted through the tube 110.

FIGS. 5A and 5B are cross-sectional views as taken at the fold 130 in the air supply tube 110 along lines 5A-5A and 5B-5B of FIGS. 4A and 4B, respectively. These figures demonstrate the relative difference in compression of the air supply tube 110 and closing of the lumen 132 without and with, respectively, insertion of the anti-occlude strip 116 of the present invention.

FIG. 5A shows air supply tube 110 having outer wall 122, and inner wall 120 forming an ovoid lumen 132, which has a minimum diameter or opening 134. It can be appreciated that minimum diameter or opening 134 is significantly less than the inner tubal diameter 128 of the air supply tube 110 when in a straightened, non-bent, and non-compressed configuration of normal operation, as shown in FIGS. 2 and 3, and it can thereby be appreciated that the air flow through the air supply tube 110 in directions 112 and 114 (shown in FIG. 4A) will be reduced substantially.

FIG. 5B shows air supply tube 110 having outer wall 122, and inner wall 120 forming a lumen 136. Lumen 136 is slightly ovoid in shape with a minimal compressed diameter 138. anti-occlude strip of the present invention may be formed with a major dimension 125A slightly less than the interior diameter of the lumen of the hollow tube, and a minor dimension that creates longitudinal apertures that allow fluids to pass through the hollow tube.

Within lumen 136 of air supply tube 110 is the anti-occlude strip 116 of the present invention, with cross-sectional shape 126, and air apertures 124A, 124B, 124C, and 124D. Air apertures 124A, 124B, 124C and 124D are patent, and allow bi-directional air flow in directions 112 and 114 (shown in FIG. 4B) through air supply tube 110.

It can be appreciated that the minimal compressed diameter 138 of lumen 136 is relatively similar to the diameter 128 of lumen 127 of the unstressed, un-flexed air supply tube 110 shown in FIGS. 2 and 3. Thereby, the relatively small reduction in the overall size of lumen 136 while under significant compression from bending at fold 130 shows the structural support of the lumen and preservation of its patency provided by the anti-occlude strip 116 of the present invention.

### Alternative Embodiments

FIGS. 6, 7, 8 and 9 show cross-sectional views of the alternative embodiments of the anti-occlude strip of the present Invention. Different shapes of the anti-occlude strip provide different flow characteristics through the air supply tube, as well as varied support profiles of the tubal structure, which would be preferable for certain medical therapy applications to those skilled in the art.

Referring now to FIG. 6, an alliterative embodiment of the anti-occlude strip 216 of the present invention inserted within an air/fluid-carrying medical therapy tube 210 having an outer wall 222, an inner wall 220, and a lumen 227, is depicted. This embodiment of the anti-occlude strip 216 of the present invention has a cross-sectional area 226 in the shape of a “Y”. The inner wall 220 and the anti-occlude strip 216 form three (3) apertures 224A, 224B, and 224C within the lumen 227 of the air/fluid-carrying medical therapy tube 210. These apertures conduct transmission of air/fluid through the air/fluid-carrying medical therapy tube 210.

FIG. 7 shows an alternative embodiment of the anti-occlude strip 316 of the present invention having a triangular-shaped cross-sectional area 326. The anti-occlude strip 316 is inserted within the lumen 327 of a medical therapy tube 310, which has an outer wall 322 and an inner wall 320. Inner wall 320 and the anti-occlude strip 316 form three (3) patent apertures 324A, 324B, and 324C, which permit flow of air or fluids through a medical therapy tube 310.

FIG. 8 shows another alternative embodiment of the anti-occlude strip 416 of the present invention having a rhomboidal or diamond-shaped cross-sectional area 426. The anti-occlude strip 416 of the present invention is located within the lumen 427 of medical therapy tube 410, which is used to carry air or fluids. Medical therapy tube 410 has an outer wall 422 and an inner wall 420. The inner wall 420 of medical therapy tube 410 and the anti-occlude strip 416 form two (2) apertures 424A and 424B through which the air or fluid is passed.

Referring to FIG. 9, another alternative embodiment of the anti-occlude strip 516 of the present invention formed with a five-pointed, star-shaped cross-sectional configuration 526, is depicted. The anti-occlude strip 516 is placed inside a lumen 527 of a medical therapy tube 510, which has an outer wall 522 and an inner wall 520. The Medical therapy tube 510 carries air or fluids. Inner wall 520 of medical therapy tube 510 and the anti-occlude strip 516 of the present invention form open apertures 524A, 524B, 524C, 524D and 524E through which the air or fluids pass.

FIG. 10 shows an alternative application of the present invention. A patient's hand 604 having an intravenous catheter 606 with an insertion point 608 into a vein in the back of the hand 604 is shown. An anti-occlude strip 616 of the present invention is within the lumen 627 of a medical-grade tube 610, which connects the intravenous catheter 606 with a medical device (not shown).

A common use of medical tubing is for delivering fluids or medications into a patient's venous system through intravenous catheterization. An intravenous catheter 606 is inserted into a vein in the patient, typically a vein in the hand or forearm, and a medical-grade tube connects catheter 606 to a medical delivery device, often an intravenous fluid bag. The distance between the patient and the medical delivery device varies widely depending on the given application, thus the length of the medical-grade tubing connecting them also varies.

A patient with intravenous catheterization may be in a stationary, sitting or recumbent position or ambulatory. Whether stationary or ambulatory, the medical tubing is at risk of bending, kinking, and compression, which will reduce or halt flow of the fluid being delivered to the patient from the medical therapy device. The result could be a medical emergency. Insertion of an anti-occlude strip 616 of the present invention into the medical tubing 610 will provide compression resistance and structural stability to keep the lumen 627 of the tube 610 patent.

In an application such as the alternative application shown in FIG. 10, the anti-occlude strip 616 of the present invention would be made from a medical-grade material, which can safely contact medical fluids and medications used in intravenous catheterization.

While there have been shown what are presently considered to be preferred embodiments and preferred applications of the present invention, it will be apparent to those skilled in the art that various changes and modifications can be made herein without departing from the scope and spirit of the invention.

1. A medical therapy device, comprising:
a tube having a lumen configured to pass air or fluids therethrough;
an anti-occlude strip having a major dimension sized to be received within said lumen and a minor dimension less than said major dimension, wherein said major dimension and said minor dimension cooperate to form at least one longitudinal aperture within said lumen.

2. The medical therapy device of claim 1, wherein said anti-occlude strip is formed with a predetermined cross-sectional shape which cooperates with said lumen to form at least one longitudinal aperture within said lumen.

3. The medical therapy device of claim 1 wherein said tube with said anti-occlude strip therein maintains patency and proper transmission of said air and fluids when said tube is compressed or bent.

4. The medical therapy device of claim 1, further comprising:
said tube has a length; and
said anti-occlude strip has a length less than said length of said tube.

5. The medical therapy device of claim 4 wherein said length of said anti-occlude strip is less than said length of said tube.

6. The medical therapy device of claim 4 wherein said length of said anti-occlude strip is equal to said length of said tube.

7. The medical therapy device of claim 1, wherein said cross-sectional shape of said anti-occlude strip forms longitudinal apertures between said strip and said tube such that said air and fluids flow therein.

8. A method of preventing occlusion of a deep vein thrombosis prevention device tube having a lumen, comprising:
providing a deep vein thrombosis prevention device comprising an inflatable garment sleeve receiving air from an air pump source via a flexible medical therapy device tube having a lumen; and
providing an anti-occlude strip within said lumen of said tube to form one or more longitudinal apertures within said tube to facilitate the passage of air from said air pump.

9. The method of preventing occlusion of a deep vein thrombosis prevention device tube of claim 8, wherein said anti-occlude strip is formed of flexible, compression-resistant material having a predetermined cross-sectional shape.

10. The method of preventing occlusion of a deep vein thrombosis prevention device tube of claim 8, further comprising the steps of:
providing a device tube having a first connector on a first end of said tube;
sizing the anti-occlude strip lengthwise similar to the device tube;
removing said first connector connectors from said first end of said device tube and inserting said anti-occlude strip into said lumen of said tube; and
replacing said first connector onto said tube to capture said anti-occlude strip within said tube.

11. (canceled)

12. A method of preventing occlusion of a medical device tube having a lumen passing fluid therethrough, comprising:
providing a medical device comprising at least one flexible medical device tube having a lumen; and
providing an anti-occlude strip within said lumen of said tube to form one or more longitudinal apertures within said tube to facilitate the passage of said fluid therethrough.

13. The medical therapy device of claim 2, wherein the predetermined cross-sectional shape is selected from the group consisting of:
cross-shaped;
‘Y’ shaped;
triangular shaped;
rhombus shaped; and
star shaped.
